(19) United States
(12) Patent Application Publication
(10) Pub. No.: US 2026/0207077 A1
(43) Pub. Date: Jul. 23, 2026

(54) ADAPTIVE REHABILITATION TRAINING SYSTEM AND METHOD BASED ON PLANTAR PRESSURE DATA

(71) Applicant: iFuturelab Group Holding Limited, TORTOLA (VG)

(72) Inventor: Daniel BROOKS, TORTOLA (VG)

(21) Appl. No.: 19/026,880

(22) Filed: Jan. 17, 2025

(51) Int. Cl. A61B 5/103; A61B 5/00; A63B 24/00

(52) U.S. Cl. CPC A61B 5/1038; A61B 5/0022; A61B 5/486; A61B 5/6807; A63B 24/0075; A61B 2505/09; A61B 2562/0247

(57) ABSTRACT

The present application discloses an adaptive rehabilitation training system and method based on plantar pressure data, the system including: a pressure sensor array for obtaining the plantar pressure data and transmitting the same synchronously to the data processing module; a data processing module for receiving, pre-processing and analyzing the plantar pressure data; a wireless communication module for transmitting the plantar pressure data and the processing result of the data processing module to an external device or a cloud; a training control module for generating a personalized rehabilitation training program based on the plantar pressure data and the processing results of the data processing module, combined with the user history data, and adaptively adjusts the rehabilitation training program; a feedback module for real-time training feedback to the user while the user is performing rehabilitation training, to help the user correct optimize movement and posture in a timely manner.

### TECHNICAL FIELD

The present disclosure relates to a technical field of smart wearable, and particularly to a n adaptive rehabilitation training system based on plantar pressure data.

### BACKGROUND

Rehabilitation medicine, as an important branch of modern medicine, is committed to helping patients recover their functions and improve their quality of life through scientific methods. In recent years, with the rapid development of artificial intelligence, smart sensing technology and smart wearable devices, emerging solutions centered on data-driven and intelligent interaction have emerged in the field of rehabilitation medicine. For example, collecting patients'physiological parameters and exercise data through sensors and analyzing these data with algorithms can provide patients with more scientific and effective training guidance.

As an important indicator of human movement and posture assessment, plantar pressure data has wide application value in rehabilitation medicine. Through plantar pressure sensors, the distribution of plantar force during exercise can be captured, thus reflecting key information such as the patient's balance ability, gait characteristics, and muscle function.

However, most of the plantar rehabilitation training systems on the market are based on preset static training programs, which lack the ability to monitor and feedback the real-time status of the patient and accurately adapt to individual differences and the dynamics of rehabilitation, making it difficult to achieve dynamic adjustment and personalized recommendations, and failing to adequately meet the diversified rehabilitation needs of patients. What's more, if the training intensity is improperly selected or the movement is not reasonably designed, it may also cause secondary injury to the patient and prolong the rehabilitation cycle.

In view of this, there is an urgent need to provide an adaptive rehabilitation training program that monitors plantar pressure data in real time and provides personalized rehabilitation guidance.

### SUMMARY

Based on the above application requirements and technical background, in order to solve the technical problem of the prior art of the inability to monitor plantar pressure data in real time and provide a personalized adaptive rehabilitation training program, the present application adopts the following technical solution:

A first aspect of the present application proposes an adaptive rehabilitation training system based on plantar pressure data, comprising a pressure sensor array, a data processing module, a wireless communication module, a training control module, and a feedback module;
the pressure sensor array comprises a weight sensor and a plurality of pressure thin-film sensors for obtaining the plantar pressure data and transmitting the same synchronously to the data processing module;
the data processing module comprises a pre-processing unit and an algorithm unit for receiving, pre-processing and analyzing the plantar pressure data to identify user gait patterns, analyze the distribution of plantar pressures, and assess the overall posture of the user;
the wireless communication module is connected to an external device or a cloud for transmitting the plantar pressure data and a processing result of the data processing module to an external device or a cloud to facilitate remote monitoring by a user;
the training control module comprises a training algorithm for generating a personalized rehabilitation training program based on the plantar pressure data and the processing results of the data processing module, combined with the user history data, and adaptively adjusts the rehabilitation training program;
the feedback module for real-time training feedback to the user while the user is performing rehabilitation training, to help the user correct optimize movement and posture in a timely manner.

Further, the weigh sensor is mounted in the center of a sole, the plurality of highly sensitive pressure thin-film sensors are embedded in key areas of the sole, the key areas comprise a heel region, a front metatarsal region, a lateral foot region, and a metatarsal arch region; wherein the weigh sensor is located below the plurality of pressure thin-film sensors.

Further, the algorithm unit comprises a first algorithm for analyzing dynamic changes in the plantar pressure data to identify the user gait patterns; the user gait patterns comprise a stride length, a stride speed, a center of gravity transfer trajectory, and a two-legged gait comparison.

Further, the algorithm unit comprises a third algorithm for assessing the overall posture of the user and determining whether the posture is correct and whether there is a posture deviation; the overall posture of the user comprising a center of gravity balance, a gait symmetry, and an abnormal pressure distribution.

Further, the wireless communication module is connected to the external device or the cloud through Bluetooth or Wi-fi, the external device including a smartphone, a tablet computer.

Further, the rehabilitation training programs comprises a training content, a training intensity, a training frequency, a training duration, and a personalized rehabilitation recommendation.

Further, the training content comprises gait conditioning, balance training or strength training; the personalized rehabilitation recommendation comprises posture adjustment, gait optimization, training intensity control.

Further, the training feedback comprises vibration feedback, sound feedback, or visual feedback.

A second aspect of the present application proposes an adaptive rehabilitation training method based on plantar pressure data, the method comprising:
obtaining the plantar pressure data and transmitting the same synchronously to a data processing module through a pressure sensor array, the pressure sensor array comprises a weight sensor and a plurality of pressure thin-film sensors;
receiving, pre-processing and analyzing the plantar pressure data to identify user gait patterns, analyze the distribution of plantar pressures, and assess the overall posture of the user through the data processing module, the data processing module comprises a pre-processing unit and an algorithm unit;
transmitting the plantar pressure data and processing results of the data processing module to an external device or a cloud to facilitate remote monitoring by a user through a wireless communication module, the wireless communication module is connected to an external device or a cloud;
generating a personalized rehabilitation training program based on the plantar pressure data and the processing results of the data processing module, combined with the user history data, and adaptively adjusts the rehabilitation training program, the training control module comprises a training algorithm;
real-time training feedback to the user while the user is performing rehabilitation training, to help the user correct optimize movement and posture in a timely manner through a feedback module.

Further, the weigh sensor is mounted in the center of a sole, the plurality of highly sensitive pressure thin-film sensors are embedded in key areas of the sole, the key areas comprise a heel region, a front metatarsal region, a lateral foot region, and a metatarsal arch region; wherein the weigh sensor is located below the plurality of pressure thin-film sensors.

Compared with the prior art, the beneficial effects of the present application are:
The system enables users to obtain real-time plantar pressure distribution data during the rehabilitation training process, comprehensively analyze their gait patterns and postures based on this data, and use machine learning algorithms to generate adaptive and dynamically adjusted rehabilitation training programs to achieve personalized rehabilitation guidance. In addition, the system provides immediate feedback through vibration, sound or visual feedback to guide the user to maintain the correct posture and movement throughout the rehabilitation training, thus enhancing the training effect and reducing potential risks. Users can select different feedback methods according to their personal preference to ensure optimal cueing during rehabilitation training. This flexible setting not only enhances the user's sense of participation, but also ensures comfort and convenience during the training process.

In practical application, this application can be used in the field of postoperative rehabilitation to help patients carry out personalized gait training after lower limb surgery, provide safe and progressive rehabilitation programs, provide real-time feedback on postural problems during training, and avoid secondary injuries; this application can be used in the field of rehabilitation for sports injuries to provide athletes or fitness enthusiasts with scientific gait analysis and postural adjustments to help them avoid undesirable movements during rehabilitation training This application can be used in the field of chronic disease management, such as diabetic foot and arthritis patients, by monitoring the distribution of plantar pressure, providing long-term rehabilitation guidance, preventing complications, and improving the quality of life; this application can be used in the field of health management for the elderly, to help the elderly to maintain the function of the lower limbs, prevent falls, and through personalized gait training, enhance their gait stability and balance ability This application can be used in hospitals and rehabilitation centers, where the system can provide doctors with detailed rehabilitation data, help professionals monitor the patient's rehabilitation progress and adjust the training plan at any time to enhance the rehabilitation effect; This application can be used in home rehabilitation care to provide remote rehabilitation support for patients who are inconvenient to travel to hospitals, with real-time feedback and remote monitoring to ensure that the rehabilitation training can be completed safely and effectively in the home environment.

By supporting remote rehabilitation, the present application can help users and healthcare organizations reduce rehabilitation costs, while reducing risks in training. Specifically, the present application reduces the need for frequent trips to hospitals or rehabilitation centers, which not only reduces the time and economic costs of rehabilitation, but also reduces the burden on medical resources, providing great convenience for patients with mobility limitations or living in remote areas; the real-time feedback mechanism and the abnormality detection function of the present application ensure that the user can timely adjust improper postures or gaits during the rehabilitation training, avoiding the incorrect training leading to The system is highly adaptable and flexible.

The system is highly adaptable and scalable, and can be flexibly configured according to the needs of different users to provide targeted rehabilitation training programs. Specifically, the system can provide adaptive training programs according to the different rehabilitation stages and needs of users, for example, for post-operative rehabilitation, the system may recommend lighter training programs, while for users recovering from sports injuries, the system will gradually increase the intensity of training to help users rebuild the flexibility of their muscles and joints; the system can be integrated with other health monitoring devices, such as heart rate monitoring, blood oxygen monitoring, etc., to further expand the dimensions of rehabilitation training, and through the integration of other health monitoring devices, such as heart rate monitoring, blood oxygen monitoring, etc. The system can integrate other health monitoring devices, such as heart rate monitoring, blood oxygen monitoring, etc., to further expand the dimension of rehabilitation training and provide more comprehensive health monitoring and rehabilitation guidance through linkage with other devices.

### BRIEF DESCRIPTION OF THE DRAWINGS

In order to more clearly explain the technical solutions in the present disclosure or the prior art, drawings required in the embodiments or the prior art will be briefly described below. Obviously, the drawings in the following description are some embodiments of the present disclosure. For those skilled in the art, other drawings may be obtained from these drawings without any creative effort.

FIG. 1 shows a schematic diagram of an adaptive rehabilitation training system based on plantar pressure data provided by the present application;

FIG. 2 is a schematic diagram of a pressure sensor array of an adaptive rehabilitation training system based on plantar pressure data provided by the present application;

FIG. 3 is a schematic diagram of a flow block diagram of an adaptive rehabilitation training system based on plantar pressure data provided by the present application.

### DETAILED DESCRIPTION OF THE EMBODIMENTS

The present application proposes an adaptive rehabilitation training system and method based on plantar pressure data, and in order to describe the present application more specifically, the technical solutions of the present application are described in detail below in connection with the accompanying drawings and specific embodiments, and it should be understood that the specific embodiments described herein are only for explaining the present application, and are not intended to limit the present application. Based on the embodiments in this application, all other embodiments obtained by a person of ordinary skill in the art without making creative labor fall within the scope of protection of this application.

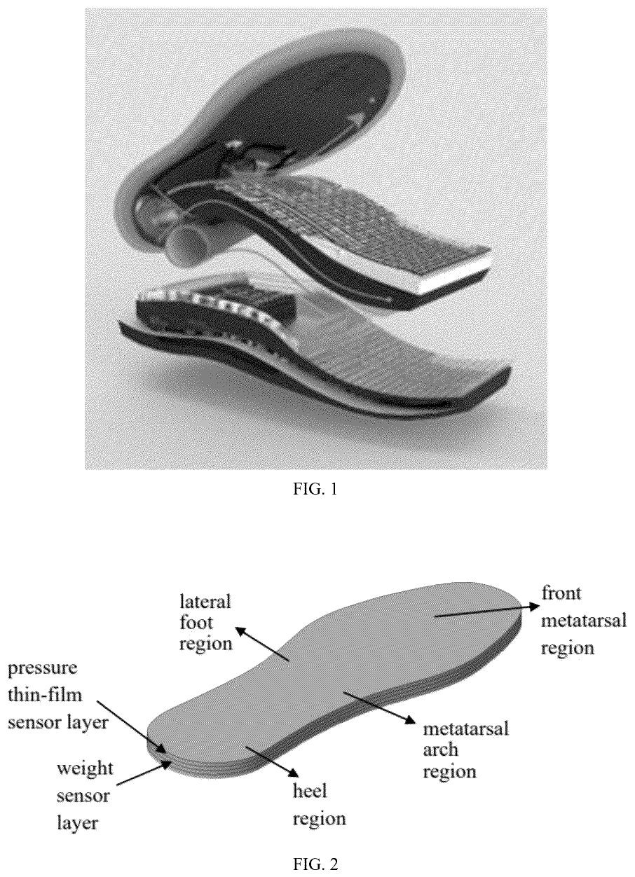

A first aspect of the present application proposes an adaptive rehabilitation training system based on plantar pressure data, as shown schematically in FIG. 1, and specifically comprising: a pressure sensor array, a data processing and analysis module, a wireless communication module, a training control module, and a feedback module;
the pressure sensor array comprises a weight sensor and a plurality of pressure thin-film sensors for obtaining the plantar pressure data and transmitting the same synchronously to the data processing module;
the pressure sensor array is shown in schematically in FIG. 2, the pressure thin-film sensors are located at the pressure thin-film sensor layer, the weight sensor is located at the weight sensor layer.

In one embodiment, the weigh sensor is mounted in the center of the sole and is responsible for measuring the overall pressure distribution on the sole of the foot in real time. Through the weight sensor, the system is able to capture the total pressure across the entire sole of the foot during walking, standing or movement, data that is important for assessing the user's center of gravity shift, weight distribution and gait stability. The weight sensor is designed to be sensitive enough to accurately capture small pressure changes and ensure that measurements are taken in real time.

In one embodiment, the plurality of highly sensitive pressure thin-film sensors are embedded in key areas of the shoe sole, the key areas include a heel region, a front metatarsal region, a lateral foot region, and a metatarsal arch region.

Wherein, the system collects plantar pressure data simultaneously from several key areas of the plantar foot, including the heel area, the front metatarsal area, the lateral foot area, and the metatarsal arch area. Plantar pressure data from these areas reflect the distribution of plantar pressures during the user's different gait phases. The wight sensor provides overall plantar pressure, while the pressure film sensor provides detailed regional pressure variations. The data collected by the sensors is transmitted at a high frequency to the system's embedded processor, ensuring real-time monitoring without delay.

The heel region is used to detect pressure changes in the heel, mainly sense the pressure when the foot follows the ground, reflecting the initial stage of the user's gait. It is worth noted that the heel is the region of maximum pressure in gait, bearing the main vertical load of the human body in standing and walking, especially at the moment of landing, the pressure is concentrated and the impact force is large;

The front metatarsal region is used to detect pressure changes in the forefoot, reflecting the user's force generation in the middle of the gait to help analyze the user's stride length and stride speed. It is worth noted that the front metatarsal provides thrust output when the user walks or runs, and the force changes are dynamic and complex;

The lateral foot region is used to detect pressure changes on the lateral side of the foot to help analyze whether there is any tilt or postural shift during the user's gait to assess the user's walking stability. It is worth noted that the lateral side of the foot mainly bears dynamic loads during walking, especially in the transition phase of the foot when the foot exerts force from the heel to the forefoot, and by analyzing the force on the lateral foot region it can help to analyze the balance of the user's gait and whether there is any problem with the foot's outward or inward roll;

The metatarsal arch region is used to detect pressure changes in the arch of the foot to help analyze the user's arch pressure-bearing situation and assess the biomechanical characteristics of the user's gait. It is worth noted that the metatarsal arch (also known as the arch of the foot) is an important cushioning structure of the sole of the foot, which can help to disperse pressure and maintain the stability of the foot, and by analyzing the force in the metatarsal arch region it can reflect the health status of the arch of the foot, which is important for evaluating the abnormality of the structure of the foot or the damage, especially for the users of flat feet or high arch of the foot.

Wherein the pressure thin-film sensors are highly sensitive and durable, capable of capturing pressure changes at every critical moment in the user's gait, and maintaining stable and accurate performance under prolonged use. Through the arrangement of these sensors, the system can capture pressure changes in different areas of the plantar foot in a comprehensive manner, which is crucial for gait analysis and adjustment of rehabilitation training.

In one embodiment, the weigh sensor is located below the pressure thin-film sensors.

The data processing module comprising a pre-processing unit and an algorithm unit for receiving, pre-processing and analyzing the plantar pressure data to identify user gait patterns, analyze the distribution of plantar pressures, and assess the overall posture of the user;

In one embodiment, the pre-processing unit is used to pre-process the received plantar pressure data, the pre-processing comprising denoising, smoothing, filtering, and correcting. External interference and abnormal data are rejected through the pre-processing to ensure the accuracy and stability of the data.

Specifically, the pre-processing step of denoising is used to eliminate noise signals generated by external interference or unwanted motion. Wherein a filter, such as a low-pass filter, enables filtering out high-frequency noise and retaining useful pressure data;

Wherein the low-pass filtering is used to pass low-frequency signals and eliminate high-frequency noise, in plantar pressure detection, real pressure signals are mainly concentrated in the low-frequency band (such as changes in gait cycles), while high-frequency components are mostly noise or interference, and the threshold of the filter used for low-pass filtering can be adjusted according to the application scenario.

Specifically, the pre-processing step of smoothing is used to further eliminate subtle fluctuations in the data, such as minor fluctuations due to gait instability or sensor sensitivity, and to generate signals with better continuity by removing short-term random variations, ensuring that the data clearly reflects the gait characteristics of the user without being affected by short-term fluctuations, and maintaining stability and continuity, comprising moving average smoothing;
wherein the moving average generates a smoother curve by calculating the average value of data points within a sliding window, which enables rapid reduction of data fluctuations and is suitable for static or low to medium frequency dynamic detection.

Specifically, the pre-processing step of correcting is used to correct anomalous data, automatically identify and reject unreasonable data points, such as sudden extreme high or low pressure values, to ensure that the final input pressure data has high accuracy and reliability.

The pre-processed data will be sent to the algorithm unit for analysis, and the algorithm in the algorithm unit is based on machine learning and deep learning techniques, which is capable of automatically recognizing the user's gait pattern, analyzing the distribution of plantar pressures and assessing the overall posture, thereby assessing the user's current rehabilitation status, and providing a basis for the system to dynamically adjust the rehabilitation training program.

In one embodiment, the algorithm unit comprises a first algorithm for analyzing dynamic changes in the plantar pressure data to identify the user gait patterns; the user gait patterns comprise stride length, stride speed, the center of gravity transfer trajectory, and two-legged gait comparison;

Specifically, the user's stride length and stride speed are calculated by the time series change of plantar pressure data, the stride length refers to the horizontal distance between two consecutive landings of the same side of the foot, reflecting the rhythmicity of the gait, and the stride length is obtained by recording the transfer of the plantar pressure from the heel region to the front metatarsal region, and calculating the horizontal displacement of the same side of the foot (e.g., the right foot) when it touches the ground twice;
the stride speed refers to the number of strides accomplished per unit of time, directly reflecting the speed of the walking, and is obtained by calculating the duration of the gait cycle, which is the time interval between two consecutive heel touches of the same foot;
the center of gravity transfer trajectory is obtained by generating the trajectory and feature extraction, and the center of gravity transfer trajectory describes how the center of gravity of the user transfers from the heel of the foot to the front metatarsal during a gait cycle, which helps to analyze the user's gait characteristics;
based on the change trajectory of plantar pressure, the system is able to identify the user's center of gravity transfer trajectory during gait, which describes how the user's center of gravity shifts from the heel to the front metatarsal bone during the gait cycle, which can help to analyze the user's gait characteristics, determine whether the user's gait is smooth or not, and whether there is any imbalance phenomenon, such as one side of the gait is biased and heavy, and then determine the effect of rehabilitation;
the two-legged gait comparison is used to detect whether the user has developed postural deviations during rehabilitation by analyzing the pressure distribution of the left and right feet, and thus detecting potential postural asymmetry problems, which is assessed by quantitatively calculating the difference between the key gait parameters of the left and right feet through the combination of the left and right foot stride lengths, touchdown time and other indexes in the gait cycle, high symmetry indicates good gait stability and coordination of movement, while low symmetry usually predicts a health problem, such as imbalance in the strength of the lower limbs or injury to the feet.

In one embodiment, the algorithm unit includes a second algorithm for analyzing pressure distributions in various regions of the plantar foot. Wherein different pressure distributions reflect different postures and movements; changes in pressure in the heel region can help the system determine whether the user is landing in the correct posture and detect whether there is uneven landing pressure; insufficient pressure in the anterior metatarsal region may mean that the user is not generating enough force during gait advancement, and that rehabilitation is progressing slowly; and the pressure distributions in the lateral plantar region and the metatarsal arch region can provide support and stability of the user's foot Pressure distribution in the lateral foot and metatarsal arch regions can provide key information about the support and stability of the user's foot, especially as excessive pressure in the lateral foot region may indicate that the user has an unstable gait, which may lead to postural imbalance.

In one embodiment, the algorithm unit includes a third algorithm for assessing the overall posture of the user and determining whether the posture is correct and whether there is a posture deviation; the overall posture of the user including center of gravity balance, gait instability, gait asymmetry and abnormal pressure distribution;

Wherein the system combines user history data and training pattern to assessing the overall posture of the user.

When the user is standing, the system can detect whether the center of gravity of his body is balanced and whether there is any bias to one side; when the user is walking, the system can assess the symmetry of the gait and determine whether the user has a tilted body or an unstable gait; the system can also detect an abnormal pressure distribution of the user, or a situation where there is an obvious deviation in the gait, such as a situation where the pressure on one side of the foot is obviously high;

once the gait imbalance or abnormal pressure distribution is detected, the system will send feedback information through the feedback module to remind the user to adjust the posture or movement to avoid secondary injuries caused by incorrect movements, and also be able to give specific adjustment suggestions, such as increasing the time of the foot on the ground or adjusting the length of the stride in order to correct the gait imbalance.

Wherein the center of gravity balance focuses on the stability of the body's center of gravity in motion or at rest. By analyzing the trajectory of the center of gravity and its related parameters, it is possible to assess balance, gait characteristics, and potential health problems;
the gait instability is a condition in which the user exhibits poor balance, abnormal gait, or unstable posture while walking. With the described gait patterns, the system can detect and recognize the unstable rhythm of gait or abnormal change in stride length, so as to identify whether the user has a problem of gait instability, which is particularly important in rehabilitation training or health monitoring of the elderly;
the gait asymmetry refers to the situation where the key parameters such as step length, step speed, touchdown time, pressure distribution and other key parameters of the left and right feet are significantly different during the user's walking process, which can be identified as an abnormal pressure state by comparing the plantar pressure data of the plantar regions of the left and right feet. The gait asymmetry may trigger chronic arthritis, imbalance of muscle strength and cause compensatory pain, etc. ;
the abnormal pressure distribution includes too much or too little pressure, the plantar pressure should change dynamically during the gait cycle and show a reasonable distribution, if the pressure in some areas is too much, it may mean that the user has gait abnormality or bad posture, and it may indicate a foot injury or plantar structural problem, such as plantar fasciitis or metatarsal fracture, if the pressure in some areas is too little or not at all, especially after a long period of time of activity, it may also mean that the user has an incorrect gait, posture or underlying plantar health problem such as a collapsed arch or unstable gait. Abnormal pressure distribution is recognized when an area is subjected to sustained high pressure above the abnormal pressure threshold (commonly associated with poor gait or forcefulness) or when an area is subjected to transient pressure above the abnormal pressure threshold (commonly associated with strenuous exercise or accidental force);

The wireless communication module is connected to an external device or a cloud and transmits plantar pressure data and processing results of the data processing module to the external device or the cloud to facilitate remote monitoring by a user;
further, the wireless communication realizes personalized adjustment of parameters in the algorithm unit by the user by transmitting user feedback inputted by the user from the external device or cloud to the data processing module.

In one embodiment, the wireless communication module is connected to an external device or the cloud through Bluetooth or Wi-fi, the external device including a smartphone, a tablet computer, and the like. The wireless connection not only enables real-time transmission of data, but also facilitates data synchronization with a health management platform or a rehabilitation monitoring system, sharing the data with medical personnel or a rehabilitation trainer, and realizing remote health monitoring and rehabilitation guidance, and the medical personnel can also monitor the changes in the plantar pressure of the patient in real time, adjust the rehabilitation plan in time, and ensure that the patient is trained within a safe range.

Specifically, connecting through Bluetooth is convenient and suitable for users'daily use at home or in rehabilitation centers; connecting through Wi-Fi is especially suitable for scenarios that require centralized management and long-term monitoring, such as hospitals and rehabilitation centers.

The wireless communication module enables the system to support remote monitoring and management functions, allowing rehabilitation doctors or medical experts to remotely view the user's rehabilitation data through the cloud, adjust the rehabilitation program according to the cloud data, and send rehabilitation suggestions to the user through the system to ensure the continuous optimization of the rehabilitation process, which is especially suitable for rehabilitation scenarios that require long-time monitoring, such as post-surgical rehabilitation or management of chronic diseases.

The training control module includes a training algorithm that generates a personalized rehabilitation training program based on the plantar pressure data and the processing results of the data processing module, combined with the user's historical data, and adaptively adjusts the rehabilitation training program;
the rehabilitation training programs include training content, training intensity, training frequency, training duration, and personalized rehabilitation recommendation to suit the user's current rehabilitation progress; the training content includes gait conditioning, balance training or strength training; the personalized rehabilitation recommendation includes posture adjustment, gait optimization, training intensity control;
the system may adjust the training content, the training intensity, the training frequency, and the training time according to the gait stability and the plantar pressure distribution of the user. For example, when the system detects that the gait and posture of the user tends to stabilize, it is determined that the user is in good condition, and the system may introduce training contents of higher difficulty, such as increasing rehabilitation programs such as gait balance training and uphill walking, or appropriately increasing the training intensity, or prolonging the training time, in order to accelerate the rehabilitation process; whereas when the system detects that the user's gait is unstable, it is determined that the user is fatigued, and the system may appropriately reduce the When the system detects that the user's gait is unstable and determines that the user is fatigued, the system may appropriately reduce the difficulty of the training content or the intensity of the training, or reduce the training time, and remind the user to rest appropriately to avoid potential injuries caused by excessive training;
the system may adjust the training content, training intensity, training frequency and training time according to the user's rehabilitation progress. For example, at the beginning of rehabilitation, the system may recommend simpler and lighter gait training, with lower training frequency and shorter training time, to ensure safety and avoid injuries caused by over-training; and as rehabilitation proceeds, the system may gradually increase the difficulty of the training, such as the introduction of balance training, up and down the stairs, etc., or gradually increase the intensity of the training, or increase the frequency of the training, or lengthen the training time, to ensure that rehabilitation is gradual and targeted, and to remind the user to rest properly. Ensure the gradual and targeted nature of rehabilitation and maximize the rehabilitation effect.

Wherein the training control module applies a deep learning algorithm to adaptively adjust the rehabilitation training program to ensure that the training program can be personalized and improved according to the individual differences of the user.
the personalized rehabilitation recommendation is based on different users'physique, rehabilitation progress and goals, this flexible adjustment mechanism can effectively improve the relevance and effect of rehabilitation.

The feedback module for real-time training feedback to the user while the user is performing rehabilitation training, to help the user correct optimize movement and posture in a timely manner, the training feedback includes vibration, sound, or visual feedback;
the system's real-time feedback mechanism ensures that users can make timely adjustments to their posture or gait during training through multiple feedback channels, improving training results and minimizing risks. The real-time feedback mechanism takes several forms, allowing the user to choose the appropriate feedback method based on personal preference;

Specifically, the vibration feedback is realized by the vibration device inside the shoe emitting a slight vibration, which is a low-interference and high-efficiency feedback method that can quickly draw the attention of the user;
the sound feedback is issued through an external device, which can provide guidance in greater detail, help the user clarify the specific requirements for adjustment, remind the user of the training movements or postures that need to be adjusted by means of voice prompts or sound, and guide the user to correct the bad postures or gaits, which is suitable for use in scenarios that require more complex adjustments;
the visual feedback is issued through an external device, which can provide the user with a graphical training progress report and adjustment suggestions, helping the user to better understand the problems in the training process and the direction of improvement, and the user can view the detailed data of the current training and the rehabilitation status through the application, obtaining more intuitive feedback information, suitable for use in a situation requiring prolonged monitoring of training.

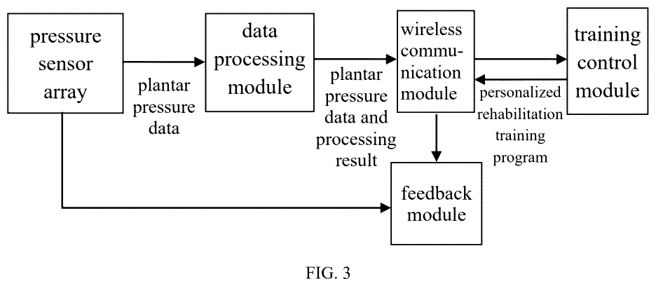

The second aspect of the present application proposes an adaptive rehabilitation training method based on plantar pressure data, a flow block diagram of the method is shown in FIG. 3, which specifically comprises:
obtaining the plantar pressure data and transmitting the same synchronously to a data processing module through a pressure sensor array, the pressure sensor array comprises a weight sensor and a plurality of pressure thin-film sensors;
receiving, pre-processing and analyzing the plantar pressure data to identify user gait patterns, analyze the distribution of plantar pressures, and assess the overall posture of the user through the data processing module, the data processing module comprises a pre-processing unit and an algorithm unit;
transmitting the plantar pressure data and processing results of the data processing module to an external device or a cloud to facilitate remote monitoring by a user through a wireless communication module, the wireless communication module is connected to an external device or a cloud;
generating a personalized rehabilitation training program based on the plantar pressure data and the processing results of the data processing module, combined with the user history data, and adaptively adjusts the rehabilitation training program, the training control module comprises a training algorithm;
real-time training feedback to the user while the user is performing rehabilitation training, to help the user correct optimize movement and posture in a timely manner through a feedback module.

Wherein the weigh sensor is mounted in the center of a sole, the plurality of highly sensitive pressure thin-film sensors are embedded in key areas of the sole, the key areas comprise a heel region, a front metatarsal region, a lateral foot region, and a metatarsal arch region; wherein the weigh sensor is located below the plurality of pressure thin-film sensors.

The foregoing is only a preferred embodiment of the present application, and is not intended to limit the present application, and the person skilled in the art should be able to realize that many examples can exist according to the basic method principles provided in the present application in combination with the actual situation, which should all be within the scope of protection of the present application without paying sufficient creative labor.

In the description of the present specification, reference is made to the terms “an embodiment”, “some embodiments”, “example”, “specific example”, or “a specific example”, “some examples”, “exemplary”, “specific examples”, or “some examples”, etc. are described to mean that the specific features, structures, materials, or characteristics described in conjunction with the embodiment or example are included in at least one embodiment or example of the present application. In this specification, schematic expressions of the above terms are not necessarily directed to the same embodiment or example. Moreover, the specific features, structures, materials, or characteristics described may be combined in a suitable manner in any one or more embodiments or examples. Moreover, without contradicting each other, those skilled in the art may combine and combine different embodiments or examples and features of different embodiments or examples described herein.

It is also noted that in this specification, relational terms such as first and second are used only to distinguish one entity or operation from another, and do not necessarily require or imply the existence of any such actual relationship or order between these entities or operations. Furthermore, the terms “including”, “comprising”, or any other variant thereof, are intended to cover non-exclusive inclusion, such that a process, method, article, or apparatus comprising a set of elements includes not only those elements, but also other elements not expressly listed, or other elements not expressly listed for the purpose of such a process, method, article, or apparatus. elements, or which are inherent to such process, method, article or equipment.

What is claimed is:

1. An adaptive rehabilitation training system based on plantar pressure data, comprising a pressure sensor array, a data processing module, a wireless communication module, a training control module, and a feedback module;
the pressure sensor array comprises a weight sensor and a plurality of pressure thin-film sensors for obtaining the plantar pressure data and transmitting the same synchronously to the data processing module;
the data processing module comprises a pre-processing unit and an algorithm unit for receiving, pre-processing and analyzing the plantar pressure data to identify user gait patterns, analyze the distribution of plantar pressures, and assess the overall posture of the user;
the wireless communication module is connected to an external device or a cloud for transmitting the plantar pressure data and a processing result of the data processing module to an external device or a cloud to facilitate remote monitoring by a user;
the training control module comprises a training algorithm for generating a personalized rehabilitation training program based on the plantar pressure data and the processing results of the data processing module, combined with the user history data, and adaptively adjusts the rehabilitation training program;
the feedback module for real-time training feedback to the user while the user is performing rehabilitation training, to help the user correct optimize movement and posture in a timely manner.

2. The adaptive rehabilitation training system of claim 1, wherein the weigh sensor is mounted in the center of a sole, the plurality of highly sensitive pressure thin-film sensors are embedded in key areas of the sole, the key areas comprise a heel region, a front metatarsal region, a lateral foot region, and a;
wherein the weigh sensor is located below the plurality of pressure thin-film sensors.

3. The adaptive rehabilitation training system of claim 2, wherein the algorithm unit comprises a first algorithm for analyzing dynamic changes in the plantar pressure data to identify the user gait patterns; the user gait patterns comprise a stride length, a stride speed, a center of gravity transfer trajectory, and a two-legged gait comparison.

4. The adaptive rehabilitation training system of claim 1, wherein the algorithm unit comprises a third algorithm for assessing the overall posture of the user and determining whether the posture is correct and whether there is a posture deviation; the overall posture of the user comprising a center of gravity balance, a gait symmetry, and an abnormal pressure distribution.

5. The adaptive rehabilitation training system of claim 1, wherein the wireless communication module is connected to the external device or the cloud through Bluetooth or Wi-fi, the external device comprising a smartphone, a tablet computer.

6. The adaptive rehabilitation training system of claim 1, wherein the rehabilitation training programs comprises a training content, a training intensity, a training frequency, a training duration, and a personalized rehabilitation recommendation.

7. The adaptive rehabilitation training system of claim 6, wherein the training content comprises gait conditioning, balance training or strength training; the personalized rehabilitation recommendation comprises posture adjustment, gait optimization, training intensity control.

8. The adaptive rehabilitation training system of claim 1, wherein the training feedback comprises vibration feedback, sound feedback, or visual feedback.

9. An adaptive rehabilitation training method based on plantar pressure data, comprising:
obtaining the plantar pressure data and transmitting the same synchronously to a data processing module through a pressure sensor array, the pressure sensor array comprises a weight sensor and a plurality of pressure thin-film sensors;
receiving, pre-processing and analyzing the plantar pressure data to identify user gait patterns, analyze the distribution of plantar pressures, and assess the overall posture of the user through the data processing module, the data processing module comprises a pre-processing unit and an algorithm unit;
transmitting the plantar pressure data and processing results of the data processing module to an external device or a cloud to facilitate remote monitoring by a user through a wireless communication module, the wireless communication module is connected to an external device or a cloud;
generating a personalized rehabilitation training program based on the plantar pressure data and the processing results of the data processing module, combined with the user history data, and adaptively adjusts the rehabilitation training program, the training control module comprises a training algorithm;
real-time training feedback to the user while the user is performing rehabilitation training, to help the user correct optimize movement and posture in a timely manner through a feedback module.

10. The plantar pressure measurement method of claim 9, wherein the weigh sensor is mounted in the center of a sole, the plurality of highly sensitive pressure thin-film sensors are embedded in key areas of the sole, the key areas comprise a heel region, a front metatarsal region, a lateral foot region, and a metatarsal arch region; wherein the weigh sensor is located below the plurality of pressure thin-film sensors.
